(19) United States
(12) Patent Application Publication
(10) Pub. No.: US 2021/0106345 A1
(43) Pub. Date: Apr. 15, 2021

(54) BONE TREATMENT GUIDE, BONE TREATMENT GUIDE SET, AND METHOD FOR USING BONE TREATMENT GUIDE

(71) Applicant: Kyoto University, Kyoto-shi, Kyoto (JP)

(72) Inventor: Yutaka KURODA, Kyoto (JP)

(73) Assignee: Kyoto University, Kyoto-shi, Kyoto (JP)

(21) Appl. No.: 16/496,373

(22) PCT Filed: Mar. 22, 2018
(86) PCT No.: PCT/JP2018/011443

§ 371 Date: Sep. 20, 2019

(30) Foreign Application Priority Data
Mar. 23, 2017 (JP) 2017-057957

(51) Int. Cl. A61B 17/17; A61B 10/02

(52) U.S. Cl. CPC A61B 17/175; A61B 17/1703; A61B 2560/0443; A61B 2560/0406; A61B 10/025

(57) ABSTRACT

A bone treatment guide, a bone treatment guide set, and a method for using a bone treatment guide are provided that enable a treatment tool, such as a trephine, to be accurately disposed relative to a bone of a patient, and that can keep a positioning member, such as a guide pin, from affecting treatment using the treatment tool. A parallel guide 5 has a positioning guide portion 13 that is to be connected to a guide pin 2, which is to be temporarily fixed to a patient's femur 101, and a treatment tool guide portion 14 configured to be able to guide a trephine 3 for treating the femur 101 toward a position at which the trephine 3 is to enter the inside of the femur 101 and in an entering direction, the treatment tool guide portion 14 being arranged in an area different from the positioning guide portion 13.

### TECHNICAL FIELD

The present invention relates to a bone treatment guide, a bone treatment guide set, and a method for using a bone treatment guide that are used when treatment, such as medical treatment, is performed on a bone of a patient.

### BACKGROUND ART

There are cases where a hole is made in a bone using a drill during artificial joint surgery and treatment of an in vivo bone, and a guide pin for guiding the drill is used at this time. Medical tools for accurately inserting the guide pin have also been developed (e.g. see Patent Document 1).

A guide pin insertion jig described in Patent Document 1 has a guide pin insertion cylinder portion for passing a guide pin therethrough. The guide pin passes through the insertion cylinder portion, and is thus inserted into a patient's tibia while being guided in an advancing direction by the insertion cylinder portion.

### CITATION LIST

### Patent Document

Patent Document 1: JP 2015-100451A

### DISCLOSURE OF THE INVENTION

### Problem to be Solved by the Invention

One known bone disease is femoral head osteonecrosis. Femoral head osteonecrosis is an intractable disease (intractable disease designated by the Ministry of Health, Labour and Welfare) that frequently occurs in people in their thirties to forties. It is known that a femoral head of 80% of patients with femoral head osteonecrosis collapses (crushes). Collapse of a femoral head makes walking difficult. Methods for treating femoral head osteonecrosis include artificial hip joint replacement surgery for replacing a patient's hip joint with an artificial hip joint. However, artificial hip joint replacement surgery requires highly invasive surgery that accompanies replacement of a hip joint, and the burden associated with the surgery is significant in terms of concern of infectious diseases and concern of dislocation, for example.

Also, performing preventive surgery before the bone head has collapsed is known as another treatment of femoral head osteonecrosis. This preventive surgery is low-invasive surgery in which a bFGF (basic fibroblast growth factor) slow-release gelatin hydrogel with slow-release properties is locally administered to a femoral head a single time. With this surgery, the effect of suppressing progress of osteonecrosis of the bone head can be expected.

As treatment for a case where the bone head has collapsed, a surgery method is conceivable in which a carrier in which a growth factor is slow-released is administered to a necrotized portion of a femoral head and the patient's own bone is implanted as autologous bone into the necrotized portion. In this surgery only the autologous bone is implanted into the necrotized portion, or gelatin hydrogel serving as a slow-release medium that contains a growth factor with a bone formation effect is directly administered, together with the autologous bone, to the necrotized portion in the bone head. It is thought that administration of a growth factor such as bFGF alone into a bone head has not been reported in or out of Japan, except for a report submitted by the inventor of the present application, as of the time the present application was filed.

In the case of autologous bone grafting, autologous bone is collected from a region other than the affected area, e.g. from the patient's ilium, and is used for implantation. At this time, a problem arises in that the portion at which the bone is collected hurts. It is favorable in terms of a reduction in surgery costs, for example, to collect autologous bone and transplant the bone in the same operation. For this reason, a method is conceivable in which autologous bone is collected from a normal portion of the lateral part of a femur which is the patient's affected area, and the collected autologous bone is provided to the inside of the femoral head.

Conventionally to collect a portion of a femur to transplant autologous bone, there is a method in which a guide pin serving as a positioning member is inserted into the femur, a tubular trephine, which is a bone-drilling tool, is inserted into the femur with the guide pin serving as an axis, and the femur is cut. The bone collected using this method has a donut shape with a hole made at the portion at which the guide pin has been disposed, and is thus fragile. Moreover, the bone collected using a trephine is collected in a narrow space between a shaft-like guide pin and a tubular trephine, and thus, the amount of bone collected is small. In addition, if the position of the guide pin that is temporarily fixed to the femur differs from the portion at which the bone is to be collected, there is also a possibility that the portion at which a hole is to be formed in the bone by the trephine will not be appropriate. For these reasons, it is favorable that the portion at which the bone is to be collected using the trephine relative to the guide pin can be set more accurately.

The foregoing problem also applies to treatment of bones other than a bone treated for femoral head osteonecrosis.

Based on the above-stated viewpoints, an object of the present invention is to provide a bone treatment guide, a bone treatment guide set, and a method for using a bone treatment guide that enable a treatment tool, such as a trephine, to be accurately disposed relative to a bone of a patient, and that can keep a positioning member, such as a guide pin, from affecting treatment using the treatment tool.

### Means for Solving the Problem

(1) A bone treatment guide according to the present invention for achieving the above-stated object includes: a positioning guide portion that is to be connected to a positioning member, which is to be temporarily fixed to a bone of a patient; and a treatment tool guide portion configured to be able to guide a treatment tool for treating the bone toward a position at which the treatment tool is to enter inside of the bone and in an entering direction, the treatment tool guide portion being arranged in an area different from the positioning guide portion.

According to this configuration, the positioning member is temporarily fixed to a bone through a manual operation or the like performed by a surgeon. Next, the positioning guide portion is connected to the positioning member, and thus the orientation and the position of the treatment tool guide portion relative to the positioning member are set. That is to say, the bone treatment guide is accurately positioned relative to the bone. Thus, the treatment tool guide portion of the bone treatment guide can guide the treatment tool such that the treatment tool advances toward an accurate position and in an accurate entering direction relative to the bone. For example, if the treatment tool is a trephine for collecting bone, the trephine can advance toward an accurate position and in an accurate advancing direction relative to the bone, and as a result, a desired portion of the femur can be collected with high position accuracy using this trephine. Furthermore, the position of the positioning guide portion differs from the position of the treatment tool guide portion. With this configuration, the positioning member connected to the positioning guide portion and the treatment tool guided by the treatment tool guide portion can be disposed in separate areas. For example, the trephine serving as a treatment tool does not need to collect bone so as to avoid a positioning member such as a guide pin. With this configuration, the bone collected using the trephine need not have a fragile shape, such as a donut shape, due to being affected by the positioning member, and furthermore, the amount of bone collected using the trephine can be further increased. Therefore, according to the present invention, a bone treatment guide can be realized that enables the treatment tool such as a trephine to be accurately disposed relative to a bone of a patient, and that can keep a positioning member such as a guide pin from affecting treatment using the treatment tool.

(2) There are cases where a shape of the positioning guide portion differs from a shape of the treatment tool guide portion.

According to this configuration, the surgeon can distinguish between the positioning guide portion and the treatment tool guide portion more reliably, due to the synergistic effect of this configuration and the configuration in which the shape of the positioning guide portion differs from the shape of the treatment tool guide portion.

(3) There are cases where both the positioning guide portion and the treatment tool guide portion are formed to have a cylindrical shape, and a diameter of the positioning guide portion is set smaller than a diameter of the treatment tool guide portion.

(4) There are cases where the bone treatment guide further includes: a holding portion to be held by a surgeon; and a connecting portion that is connected to the positioning guide portion, the treatment tool guide portion, and the holding portion.

(5) There are cases where a pair of the positioning guide portions are provided, and the pair of positioning guide portions are connected to the treatment tool guide portion on respective sides of the treatment tool guide portion in a width direction perpendicular to a longitudinal direction of the treatment tool guide portion.

According to this configuration, the positioning guide portions are connected on the two respective sides of the treatment tool guide portion in the width direction. Accordingly when connecting the positioning guide portions to the positioning member, the surgeon can selectively use either one of the pair of positioning guide portions provided on the two sides of the treatment tool guide portion in the width direction. For this reason, the same bone treatment guide can be used even if the positioning member is temporarily fixed to the bone at either of the positions on the two sides, in the width direction, of the treatment tool that is to enter the inside of the bone, relative to the position at which the treatment tool is to enter the inside of the bone. For example, in the case where the treatment tool is a trephine for collecting bone, the same bone treatment guide can be used even if a positioning member such as a guide pin is temporarily fixed to a femur at any position in the width direction of the trephine, relative to the trephine. As a result, the same bone treatment guide can be used even if the bone treatment guide is used in surgery on either the left leg or the right leg.

(6) There are cases where the bone treatment guide further includes a replaceable member having a tubular shape that is replaceably attached to the treatment tool guide portion by being detachably inserted into the treatment tool guide portion that is formed to have a tubular shape, wherein the treatment tool is guided toward the position at which the treatment tool is to enter the inside of the bone and in the entering direction, by being inserted into the replaceable member attached to the treatment tool guide portion.

According to this configuration, when the treatment tool is guided to enter the inside of the bone by the treatment tool guide portion, the treatment tool is inserted into the replaceable member attached to the treatment tool guide portion. For this reason, the treatment tool can be prevented from directly coming into contact with the treatment tool guide portion, and thus causing damage such as wear. Even if the replaceable member is damaged, e.g. is worn when the bone treatment guide is used, only the replaceable member can be readily replaced. For this reason, the treatment tool guide portion can be repeatedly used simply by replacing the replaceable member, without the treatment tool guide portion being broken.

(7) A bone treatment guide set according to the present invention for achieving the above-stated object includes: the above-described bone treatment guide; and an image guide, wherein the image guide includes: a positioning member corresponding portion for indicating an arrangement of the positioning member; and a treatment tool corresponding portion for indicating an arrangement of the treatment tool.

According to this configuration, the surgeon can envision the arrangement of the positioning member and the treatment tool for when the positioning member and the treatment tool have entered the bone of the patient, by holding the positioning member corresponding portion and the treatment tool corresponding portion over the surface of the patient, for example. As a result, the surgeon can more accurately arrange the positioning member and the treatment tool relative to the bone of the patient. Specifically, if the image guide is displayed on a monitor of a radiographic device, such as an X-ray device, a state where the bone of the patient is overlapped with the image guide is displayed on the monitor allowing the surgeon to more accurately understand the path that the positioning member and the treatment tool will take.

(8) There are cases where a shape of an outer edge of the positioning member corresponding portion corresponds to a shape of an outer edge of the positioning member, and a shape of an outer edge of the treatment tool corresponding portion corresponds to a shape of an outer edge of the treatment tool.

According to this configuration, the surgeon can more accurately envision the positions of the positioning member and the treatment tool for when the positioning member and the treatment tool have entered the bone of the patient, by displaying the positioning member corresponding portion and the treatment tool corresponding portion on the monitor of the radiographic device, for example.

(9) There are cases where the bone treatment guide and the image guide are fixed to each other and are formed into an integrated member.

According to this configuration, the surgeon can simultaneously grip the bone treatment guide and the image guide, and thus can more readily handle the bone treatment guide. Furthermore, since the relative positions of the bone treatment guide and the image guide are fixed, time and effort involved in adjusting the relative positions of the bone treatment guide and the image guide can be saved.

(10) There are cases where the bone treatment guide set further includes a connecting bar for connecting the bone treatment guide and the image guide to each other, wherein the bone treatment guide and the image guide are detachably connected to each other via the connecting bar.

According to this configuration, when an image guide is required, the bone treatment guide and the image guide can be connected by the connecting bar. Meanwhile, if the image guide is not required, the bone treatment guide can be used alone by canceling the connection between the bone treatment guide and the image guide via the connecting bar; and removing the image guide alone or together with the connecting bar from the bone treatment guide.

(11) A method for using a bone treatment guide according to the present invention for achieving the above-stated object includes: a first check step of checking a path of a positioning member using a positioning member corresponding portion of an image guide that includes: the positioning member corresponding portion for indicating an arrangement of the positioning member, which is to be temporarily fixed to a bone of a patient; and a treatment tool corresponding portion for indicating an arrangement of a treatment tool for treating the bone; a position setting step of setting a position of the above-described bone treatment guide relative to the bone while connecting the positioning member that has been temporarily fixed to the bone to the positioning guide portion of the bone treatment guide; a second check step of checking a path of the treatment tool using the treatment tool corresponding portion of the image guide; and a bone collection step of causing the treatment tool to enter inside of the bone while guiding the treatment tool using the treatment tool guide portion, and collecting a portion of the bone using the treatment tool.

According to this configuration, in the first check step, the surgeon envisions a predicted position of the positioning member relative to the bone, using an arrangement image of the positioning member indicated by the positioning member corresponding portion. The surgeon then determines the arrangement of the positioning member while referencing this image. Furthermore, in the position setting step, the surgeon can set the position of the bone treatment guide at an accurate position relative to the bone by connecting the positioning guide portion of the bone treatment guide to the positioning member. Next, in the second check step, the surgeon envisions a predicted path of the treatment tool relative to the bone, using an arrangement image of the treatment tool indicated by the treatment tool corresponding portion. The surgeon can then reference this image to make the treatment tool enter the inside of the bone and collect a portion of the bone using the treatment toot while guiding the treatment tool using the treatment tool guide portion. Thus, by using the bone treatment guide and the image guide, the surgeon can collect a desired portion of the bone of the patient at an accurate position, and can keep the positioning member from affecting the collection of bone using the treatment tool.

(12) There are cases where the method for using a bone treatment guide further includes an introduction step of introducing a bone composition into the bone through a hole portion formed in the bone during the bone collection step.

According to this configuration, the surgeon can introduce a bone composition into a more appropriate area through the hole portion formed in the bone. As a result, the bone can heal more quickly.

### Effects of the Invention

According to the present invention, a treatment tool such as a trephine can be accurately disposed relative to the bone of the patient, and strong bone can be collected using the treatment tool.

### BRIEF DESCRIPTION OF THE DRAWINGS

FIG. 1 is a schematic side view of constituent elements of a bone treatment guide set according to an embodiment of the present invention.

FIG. 2 shows an example the bone treatment guide. FIG. 2(A) is a perspective view, and FIG. 2(B) is a side view.

FIG. 3 shows an example of the bone treatment guide. FIG. 3(A) is a bottom view and FIG. 3(B) is a front elevational view.

FIG. 4 shows an image guide of the bone treatment guide set. FIG. 4(A) is a plan view of the image guide, and FIG. 4(B) is a side view of the image guide.

FIG. 5 is a schematic plan view showing an example of a state where a patient's femur is treated using the bone treatment guide set, partially showing a cross-sectional view FIG. 6 is a flowchart showing an example of a surgical procedure using the bone treatment guide set.

FIGS. 7(A) and 7(B) show an example of a surgical procedure using the bone treatment guide set. FIG. 7(A) illustrates step S1, and is a plan view of a main part showing a state where the image guide is aligned with the front side of the patient. FIG. 7(B) illustrates step S1, and shows an image appearing on a monitor of a radiographic device.

FIGS. 8(A) and 8(B) show an example of a surgical procedure using the bone treatment guide set. FIG. 8(A) illustrates step S2, and FIG. 8(B) illustrates step S3.

FIGS. 9(A) and 9(B) show an example of a surgical procedure using the bone treatment guide set. FIG. 9(A) illustrates step S4, and is a plan view of a main part showing a state where the image guide is aligned with the front side of the patient. FIG. 9(B) illustrates step S4, and shows an image appearing on the monitor of the radiographic device.

FIGS. 10(A) and 10(B) show an example of a surgical procedure using the bone treatment guide set. FIG. 10(A) illustrates step S5, and FIG. 10(B) illustrates step S6.

FIGS. 11(A) and 11(B) show an example of a surgical procedure using the bone treatment guide set. FIG. 11(A) illustrates step S7, and FIG. 11(B) illustrates step S8.

FIG. 12(A) is a side view showing a main part of a modification of the present invention, and FIG. 12(B) is a side view showing a main part of a further modification of the modification shown in FIG. 12(A).

FIG. 13(A) is a side view showing a main part of a modification based on the configuration in FIG. 12(A) of the present invention, and FIG. 13(B) is a side view showing a main part of a modification based on the configuration in FIG. 12(B) of the present invention.

FIG. 14 shows a main part of a yet another modification of the present invention. FIG. 14(A) is a plan view of the image guide, and FIG. 14(B) is a side view of the image guide in FIG. 14(A).

FIG. 15 is a plan view showing a main part of a yet another modification of the present invention.

FIG. 16 is a front elevational view of the bone treatment guide shown in FIG. 15.

FIG. 17 is a side view of the bone treatment guide shown in FIG. 15.

### DESCRIPTION OF EMBODIMENTS

A mode for carrying out the present invention will be described below with reference to the drawings. In this embodiment, a description will be given while taking, as an example, the case where a bone treatment guide set 1 is used to treat a patient with femoral head osteonecrosis. More specifically this embodiment employs a configuration in which the bone treatment guide set 1 makes it possible to form a hole in a patient's femur 101 and collect bone, as well as treat a collapsed portion at the same time. A surgeon then collects a portion of the femur 101 as autologous bone 105, and provides only this autologous bone 105 or a bone composition 109 that contains the autologous bone 105 to a collapsed portion 106 of a femoral head 103, using the bone treatment guide set 1.

The bone treatment guide set 1 is used to more accurately treat a bone of a patient (the femur 101 in this embodiment).

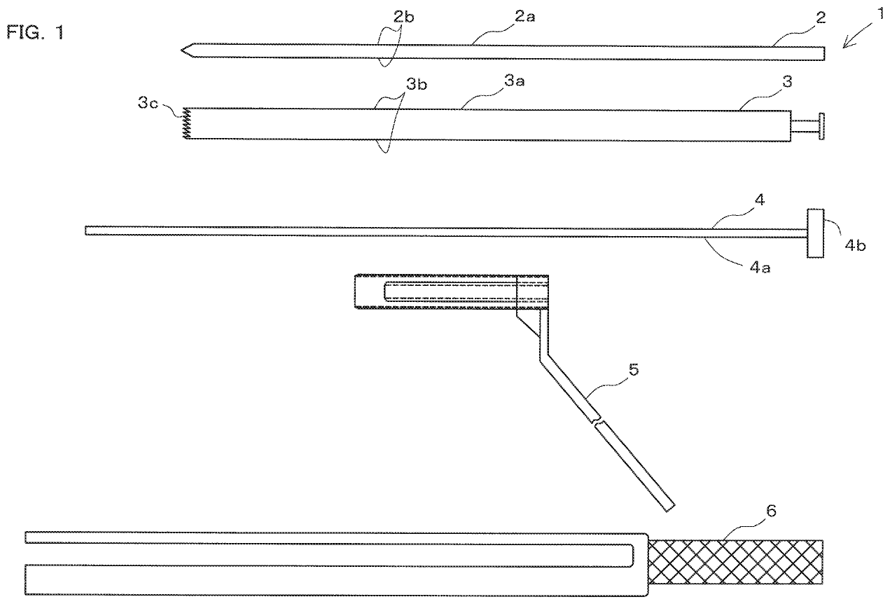

As shown in FIG. 1, the bone treatment guide set 1 includes a guide pin 2, a trephine 3, a tamper 4, a parallel guide 5, and an image guide 6.

The guide pin 2 is an example of a positioning member according to the present invention. The trephine 3 is an example of a treatment tool for treating a bone according to the present invention. The parallel guide 5 is an example of a bone treatment guide according to the present invention.

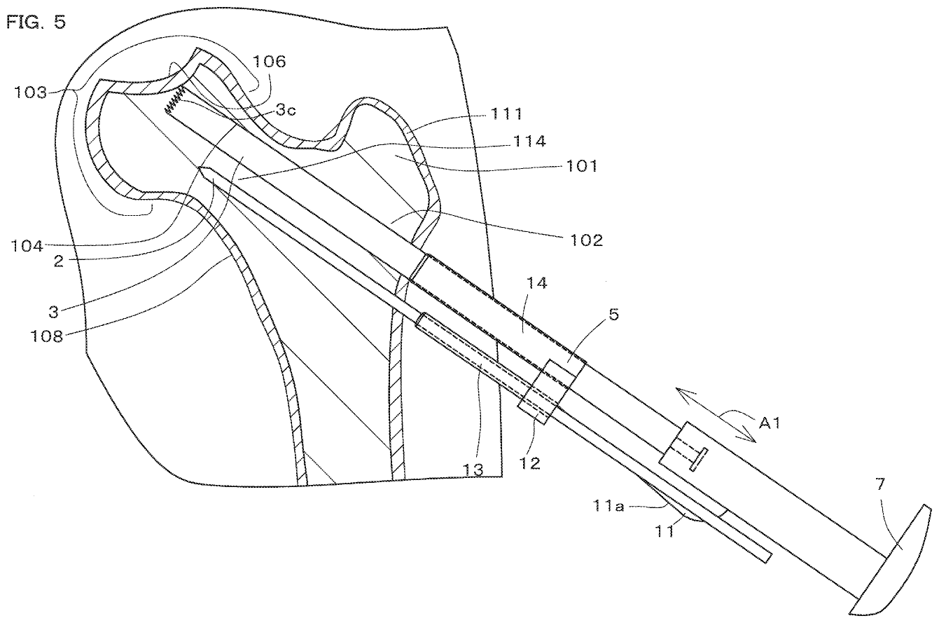

Next, the guide pin 2 and the trephine 3 will be described with reference to FIG. 5. The guide pin 2 is a member that is inserted from a lateral part (right portion) 102 of the femur 101 toward a medial part 108 thereof and is then temporarily fixed. The guide pin 2 is formed to have a cylindrical shaft shape with a leading end that is pointed to form a cone shape. The thickness of the guide pin 2 depends on the material, but is preferably 0.5 mm or more to prevent the guide pin 2, when being inserted into a bone, from bending and not being able to be inserted into the bone. Meanwhile, if the guide pin 2 is too thick, it takes time for a hole formed by the guide pin 2 to spontaneously recover, and therefore the thickness of the guide pin 2 is preferably 4 mm or less. An example of a more preferable thickness range is 1.5 mm to 3 mm. The length of the guide pin 2 also depends on the physique of the patient, but is preferably 15 to 35 cm, and more preferably 20 to 30 cm.

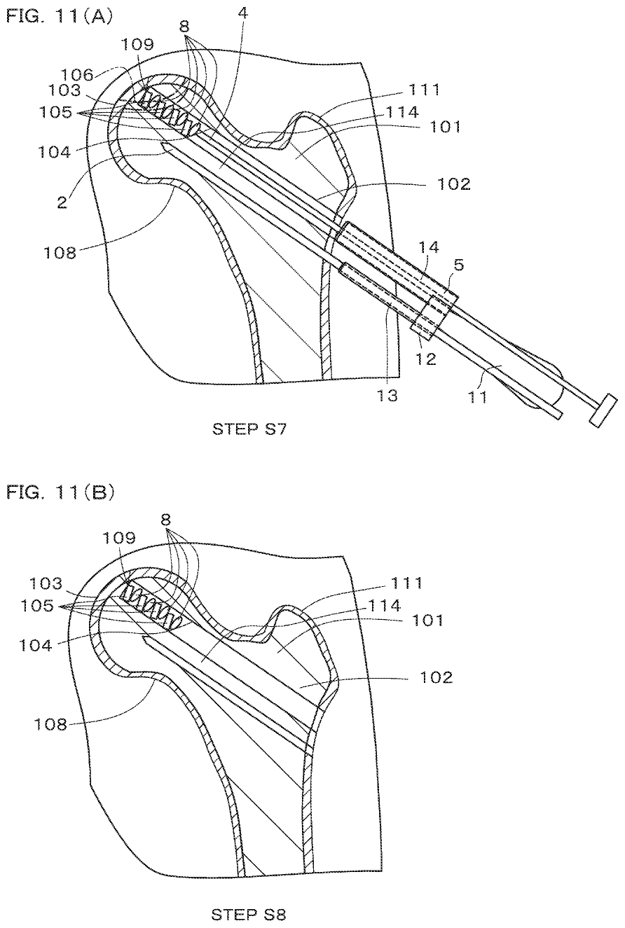

The trephine 3 is used to cut the patient's femur 101 and collect a portion thereof. Also, the trephine 3 forms an access hole portion 104 for performing treatment of the femur 101 from the femoral head 103 to a femoral neck 114 of the femur 101, by collecting a portion of the femur 101. In this embodiment, the trephine 3 advances from an outer side (right side of the diagram) of the lateral part 102 of the femur 101 and cuts the femur 101. The trephine 3 is formed to have a cylindrical shape as a whole. The inside diameter of the trephine 3 is set as appropriate in accordance with the size of the femur 101 or the like, and is set to 4 mm to 16 mm, for example, preferably 6 mm to 13 mm, and more preferably about 8 mm to 11 mm. The wall thickness of the cylindrical portion of the trephine 3 is about 0.5 mm to 1 mm, and thus, the outside diameter of the trephine 3 is 5 mm to 17 mm, preferably 7 mm to 14 mm, and more preferably 9 mm to 12 mm. A blade portion 3c is formed at a leading end of the trephine 3. This blade portion 3c cuts the femur 101 as a result of the trephine 3 being applied to the femur 101 while being rotated by a rotating tool, such as an electric drill 7. After this cutting operation has been completed, the trephine 3 is removed from the femur 101, and thus, a portion of the femur 101 that has entered the inside of the trephine 3 is collected as the autologous bone 105 (see FIG. 11(A) and FIG. 11(B)).

Next, the tamper 4 will be described with reference to FIGS. 1 and 5. The tamper 4 is a member that is used to introduce, through the access hole portion 104, a bone composition 109 (not shown in FIGS. 1 and 5), such as a gelatin gel in which the autologous bone 105 and a growth factor are slow-released, into the femoral head 103. Also, if a portion of the femoral head 103 has collapsed and a collapsed portion 106 has formed, the tamper 4 can push up the collapsed portion 106 from the inner side of the femoral head 103.

The tamper 4 has a shaft portion 4a that extends in an elongated manner, and a holding portion 4b that is fixed to a base end portion of the shaft portion 4a.

The shaft portion 4a is formed to have a cylindrical shaft shape, for example, and has an overall length that is greater than the overall length of the access hole portion 104 and an outside diameter that is smaller than the inside diameter of the trephine 3. The outside diameter of the shaft portion 4a need only be smaller than the inside diameter of the trephine 3 by 0.1 mm or more, but is typically 2 mm to 5 mm. Note that a leading end of the shaft portion 4a may be formed to have a curved hook-like shape such that the bone composition 109 can be readily pushed into the access hole portion 104 in the femur 101. The holding portion 4b is formed to have a shape that can be held in a surgeon's hand, and is formed to have a disk shape, for example.

The parallel guide 5 is a member capable of guiding the trephine 3 toward the position at which the trephine 3 is to enter the inside of the femur 101, and in the advancing direction. Particularly the parallel guide 5 guides the trephine 3, in a state of being positioned on the femur 101 via the guide pin 2. The parallel guide 5 is used to position the trephine 3 such that the trephine 3 comes into contact with the lower side of a greater trochanter 111 of the lateral part 102 of the patient's femur 101, and to orient the trephine 3 such that the trephine 3 passes through a center portion of the femoral neck 114 toward the collapsed portion 106. This embodiment will give a description while taking, as an example, a mode in which the parallel guide 5 is used in surgery on the left leg. In the case where the parallel guide 5 is used in surgery on the right leg, this parallel guide 5 has a shape (arrangement) that is a mirror, in the left-right direction of the patient, of the shape (arrangement in FIG. 5) of the parallel guide 5 when used in surgery on a patient.

The parallel guide 5 is made by combining a plurality of members with each other, or by means of integral molding. The material of the parallel guide 5 is not particularly limited as long as the material is processable and there is no concern of breakage when in use, and examples of the material may include metal and carbon fiber. The parallel guide 5 is formed to have a size that can be held in a surgeon's hand, and has, for example, a size corresponding to about two fists of a person.

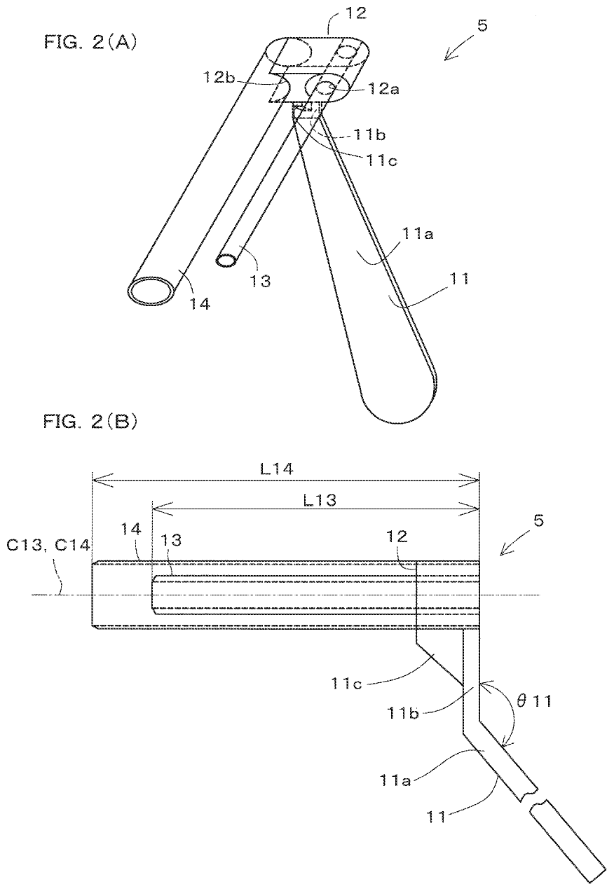

Next, the parallel guide 5 will be described with reference to FIGS. 2(A)2 (B), 3(A), and 3(B). The parallel guide 5 has a holding portion 11 that is to be held by the surgeon, a connecting portion 12, a positioning guide portion 13, and a treatment tool guide portion 14.

The holding portion 11 has a holding portion body 11a, and an extending portion 11b that is continuous with a base end portion of the holding portion body 11a.

The holding portion body 11a is formed to have a flat plate shape, for example. An end portion of the holding portion body 11a is tapered such that the width thereof increases from a base end portion toward a leading end portion of the holding portion 11, and a leading end portion of the end portion is formed to have a semicircular shape. The holding portion body 11a is inclined relative to the extending portion 11b as viewed from a side. The extending portion 11b is formed to have a rectangular flat plate shape. The extending portion 11b is arranged perpendicular to the direction in which the positioning guide portion 13 extends. A plate-shaped reinforcing portion 11c is fixed to the extending portion 11b and the connecting portion 12. The reinforcing portion 11c is arranged perpendicular to the extending portion 11b, and deformation of the extending portion 11b relative to the connecting portion 12 is suppressed. By setting an inclination angle 611 of the holding portion body 11a relative to the extending portion 11b, the orientation of the positioning guide portion 13 and the orientation of the treatment tool guide portion 14 when the surgeon holds the holding portion body 11a can beset. The inclination angle 611 of the holding portion body 11a relative to the extending portion 11b is set such that the surgeon can readily operate the parallel guide 5 in the direction in which the femoral head 103 protrudes obliquely relative to the bone axis direction in the femur 101, without the surgeon's hand that is holding the holding portion body 11 coming into contact with the patient's leg. Specifically the inclination angle θ11 is set to 120° to 160°, for example, preferably 130° to 150°, and more preferably about 140°. Note that the holding portion 11 need only have a shape capable of being held by the surgeon, and the specific shape thereof is not limited. The connecting portion 12 is continuous with the extending portion 11b, which is the base end portion of the holding portion 11.

The connecting portion 12 is a portion that is connected to the positioning guide portion 13, the treatment tool guide portion 14, and the holding portion 11, and functions as a base portion of the parallel guide 5. In this embodiment the connecting portion 12 is formed to have a block shape that surrounds a base end portion of the positioning guide portion 13. An upper face and a bottom face of an intermediate portion of the connecting portion 12 in the width direction W1 are formed into flat faces parallel to the width direction W1. The connecting portion 12 supports the base end portion of the positioning guide portion 13, and also supports a base end portion of the treatment tool guide portion 14. In this embodiment, the positioning guide portion 13 and the treatment tool guide portion 14 are arranged next to each other in parallel in the width direction W1.

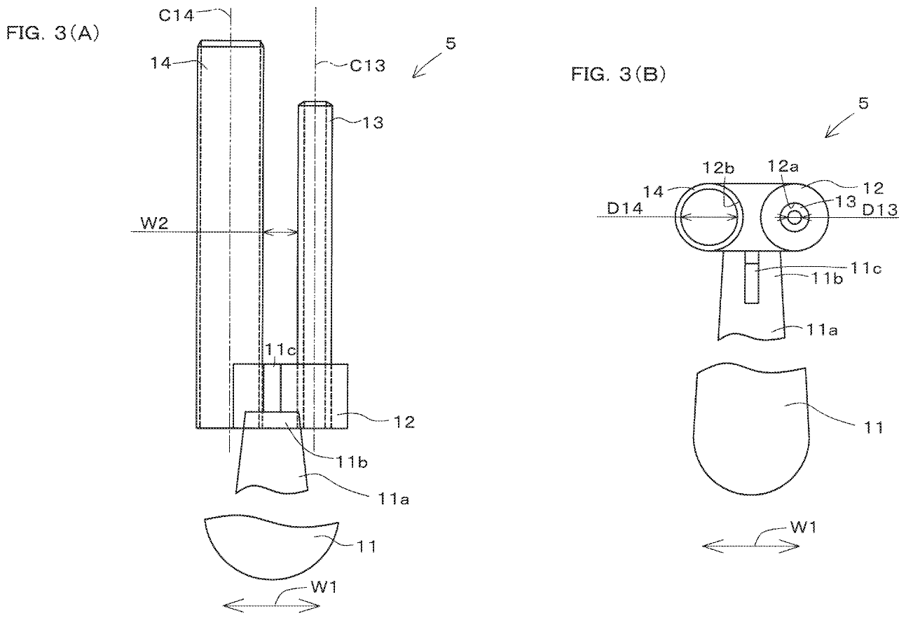

The positioning guide portion 13 is provided as a portion that is to be connected to the guide pin 2, as shown in FIG. 5. In this embodiment, the positioning guide portion 13 is formed to have an elongated cylindrical shape. The base end portion of the positioning guide portion 13 is fitted to an open hole portion 12a, which is formed in the connecting portion 12, as shown in FIGS. 3(A) and 3(B), and is fixed to this open hole portion 12a by means of welding or the like. The shape of an inner-circumferential face of the positioning guide portion 13 corresponds to the shape of an outer-circumferential face of the guide pin 2. More specifically the inside diameter D13 of the inner-circumferential face of the positioning guide portion 13 is provided with play, and is set greater than the outside diameter of the guide pin 2 by about 0.1 mm to 0.5 mm, or more preferably by 0.2 to 0.4 mm. This shape of the positioning guide portion 13 makes it possible to smoothly pass the guide pin 2 therethrough manually and suppresses rattling between the positioning guide portion 13 and the guide pin 2. Although the overall length L13 (i.e. the length in the longitudinal direction, the length in the axial direction) of the positioning guide portion 13 also depends on the physique of the patient, if the overall length L13 is too large, it will be difficult to perform fine operations, and the tool will also be heavier. On the other hand, if the overall length L13 is too small, the surgeon's hand that is holding the holding portion 11 will come into contact with the patient, which is inconvenient. For this reason, the overall length L13 of the positioning guide portion 13 is preferably 5 to 15 cm, and more preferably 8 to 12 cm.

The treatment tool guide portion 14 is used to position the trephine 3 such that the leading end of the trephine 3 comes into contact with the lower side of the greater trochanter 111 of the lateral part 102 of the patient's femur 101, and orient the trephine 3 such that the trephine 3 passes through the center portion of the femoral neck 114 toward the collapsed portion 106, as is obvious from FIG. 5. In this embodiment, the treatment tool guide portion 14 is arranged parallel to the positioning guide portion 13.

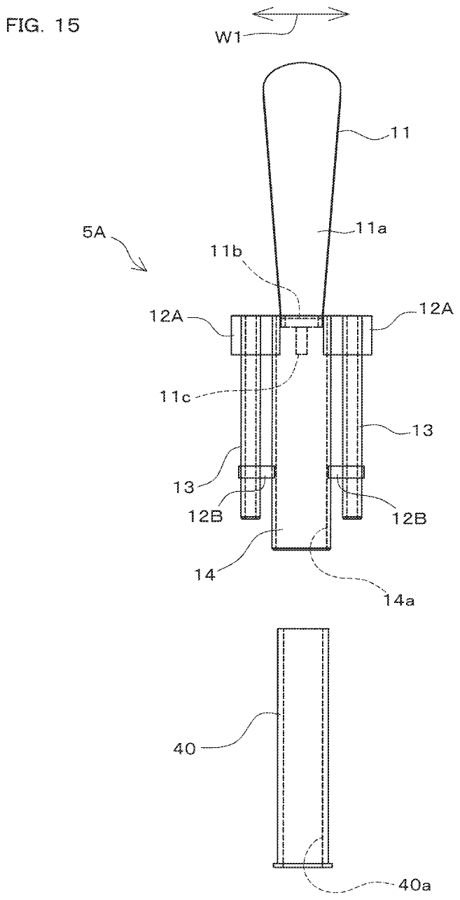

The treatment tool guide portion 14 is provided as a portion that can guide the trephine 3 toward the position at which the trephine 3 is to enter the inside of the femur 101, and in the advancing direction, and that is arranged in an area different from the positioning guide portion 13, as is obvious from FIGS. 2(A), 3(B), and 5. The treatment tool guide portion 14 is connected to the trephine 3 as a result of the trephine 3 being passed through the treatment tool guide portion 14. In this embodiment, the treatment tool guide portion 14 is formed to have an elongated cylindrical shape, and is fixed to the connecting portion 12. In this embodiment, the base end portion of the treatment tool guide portion 14 is fixed to the connecting portion 12 by means of welding or the like, in a state where the base end portion of the treatment tool guide portion 14 is arranged along a joint face 12b, which is one side face of the connecting portion 12 in the width direction W1 and has an arc-shaped recess that matches the shape of an outer-circumferential face of the base end portion of the treatment tool guide portion 14. The shape of an inner-circumferential face of the treatment tool guide portion 14 corresponds to the shape of an outer-circumferential face of the trephine 3. More specifically, the inside diameter D14 of the inner-circumferential face of the treatment tool guide portion 14 is provided with play, and is set greater than the outside diameter of the trephine 3 by about 0.1 mm to 0.5 mm, and more preferably by 0.2 mm to 0.4 mm. This shape of the treatment tool guide portion 14 enables the trephine 3 to be smoothly passed therethrough manually, and suppresses rattling between the treatment tool guide portion 14 and the trephine 3. Note that, in the case where the parallel guide 5 includes a tubular replaceable member 40 as in a later-described modification illustrated in FIG. 15, play is provided between the outside diameter of the trephine 3 and the inside diameter of the replaceable member 40, and play is provided between the outside diameter of the replaceable member 40 and the inside diameter D14 of the inner-circumferential face of the treatment tool guide portion 14. In this case, for example, the inside diameter of the replaceable member 40 is set greater than the outside diameter of the trephine 3 by about 0.01 mm to 0.3 mm, and more preferably by about 0.05 mm to 0.15 mm. The inside diameter D14 of the inner-circumferential face of the treatment tool guide portion 14 is set greater than the outside diameter of the replaceable member 40 by about 0.01 mm to 0.3 mm, and more preferably by about 0.05 mm to 0.15 mm.

Although the overall length (i.e. the length in the longitudinal direction, the length in the axial direction) L14 of the treatment tool guide portion 14 also depends on the physique of the patient, if the overall length L14 of the treatment tool guide portion 14 is too large, it will be difficult to perform fine operations, and the tool will also be heavier. On the other hand, if the overall length L14 is too small, the surgeon's hand that is holding the holding portion 11 will come into contact with the patient, which is inconvenient. For this reason, the overall length L14 of the treatment tool guide portion 14 is preferably 5 to 15 cm, and more preferably 8 to 12 cm.

In this embodiment, the shape of the positioning guide portion 13 differs from the shape of the treatment tool guide portion 14, as shown in FIGS. 1, 2(A), 3(B), and 5. More specifically, the inside diameter (diameter) D13 of the positioning guide portion 13 is set smaller than the inside diameter (diameter) D14 of the treatment tool guide portion 14. The inside diameter D13 of the positioning guide portion 13 need only be greater than the outside diameter of the guide pin 2 by about 0.1 mm, and is 1 mm to 4 mm, for example, and more preferably 1.5 to 2.5 mm. Meanwhile, the inside diameter D14 of the treatment tool guide portion 14 need only be greater than the outside diameter of the trephine 3, and is 5 to 17 mm, for example, more preferably 7 to 14 mm, and more preferably 9 to 12 mm.

The treatment tool guide portion 14 and the positioning guide portion 13 extend in the same orientation, parallel to each other from the connecting portion 12, as shown in FIGS. 2(B) and 5. In this embodiment, the center axis C13 of the positioning guide portion 13 is substantially parallel to the center axis C14 of the treatment tool guide portion 14 as viewed from a side (FIG. 2(B)). Note that these center axes C13 and C14 may be shifted from each other as viewed from a side. Meanwhile, the length L14 of the treatment tool guide portion 14 is set greater than the length L13 of the positioning guide portion 13. Thus, the amount by which the treatment tool guide portion 14 protrudes from the connecting portion 12 is greater than the amount by which the positioning guide portion 13 protrudes from the connecting portion 12. This configuration makes it possible to abut the leading ends of the guide portions 13 and 14 together against the lower side of the greater trochanter 111 of the lateral part 102 of the femur 101, and to orient the treatment tool guide portion 14 such that it passes through the center portion of the femoral neck 114 toward the collapsed portion 106.

In this embodiment, the position of the center axis C13 of the positioning guide portion 13 is shifted from the position of the center axis C14 of the treatment tool guide portion 14 in the width direction W1 of the holding portion 11, as clearly shown in FIG. 3(A). Due to this arrangement, a predetermined gap W2 is provided between the positioning guide portion 13 and the treatment tool guide portion 14 so that they do not overlap in any area as viewed from the front (FIG. 3(B)). If the width W2 is too small, there may be cases where a hole formed by the trephine 3 is too close to a hole formed by the guide pin 2, and the strength of the bone may decreases. On the other hand, if the width W2 is too large, the guide pin 2 may come loose from the femoral neck 114 (see FIG. 5), and thus, the width W2 is usually 4 to 12 mm, and preferably 5 to 10 mm.

The positioning guide portion 13 is fixed in a state of being inserted into the open hole portion 12a in the connecting portion 12, as shown in FIG. 2(A). Meanwhile, the treatment tool guide portion 14 is fixed to the connecting portion 12, in a state of being arranged along the arc-shaped recessed joint face 12b of the connecting portion 12. Thus, the shape of the portion of the connecting portion 12 that fixes the positioning guide portion 13 may differ from the shape of the portion of the connecting portion 12 that fixes the treatment tool guide portion 14.

Next, the image guide 6 will be described with reference to FIGS. 1, 4(A), 4(B), and 5. The image guide 6 is used to visually check the arrangement of the guide pin 2 and the trephine 3 when an affected area (hip joint) of the patient is observed using a radiographic device, such as an X-ray device, for seeing the affected area using a radioscopic method during surgery. The image guide 6 is made of a material that can be visually recognized through the radiographic device, such as metal, and is used in a state of being held over the surface of the patient, outside the patient's body.

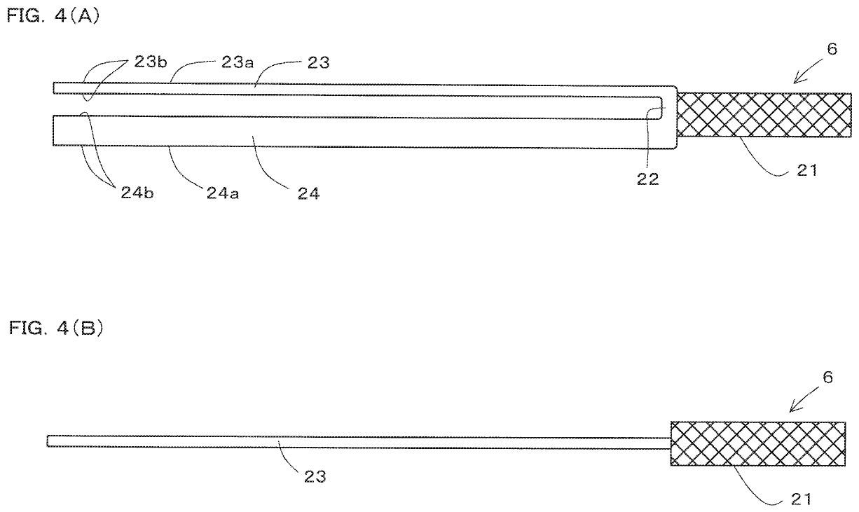

The image guide 6 has a holding portion 21 that is to be held by the surgeon, a connecting portion 22, a positioning member corresponding portion 23 for indicating the arrangement of the guide pin 2, and a treatment tool corresponding portion 24 for indicating the arrangement of the trephine 3, as shown in FIG. 4(A).

The holding portion 21 is formed to have a shape suitable for gripping by the surgeon, such as a circular column shape. An outer-circumferential face of the holding portion 21 may be subjected to non-slip work, such as knurling, as shown in FIG. 4. The shape of the holding portion 21 need only be a shape that can be held by a surgeon, and the specific shape thereof is not limited. The connecting portion 22 is continuous with a leading end portion of the holding portion 21.

The connecting portion 22 is a portion that is connected to the positioning member corresponding portion 23, the treatment tool corresponding portion 24, and the holding portion 21, and functions as a base portion of the image guide 6. In this embodiment, the connecting portion 22 is formed to have a plate shape that is fixed to one end face of the holding portion 21 and extends in the radial direction of the holding portion 21. The connecting portion 22 supports a base end portion of the positioning member corresponding portion 23, and also supports a base end portion of the treatment tool corresponding portion 24. In this embodiment, the positioning member corresponding portion 23 and the treatment tool corresponding portion 24 are arranged next to each other in the width direction of the holding portion 21. Also, in this embodiment, the connecting portion 22, the positioning member corresponding portion 23, and the treatment tool corresponding portion 24 are formed into an integrally molded piece, which is formed by cutting a single metal plate.

The positioning member corresponding portion 23 displays an image that simulates the guide pin 2 when viewed through a radiographic device. The positioning member corresponding portion 23 is formed to have an elongated rectangular flat plate shape. In this embodiment, when the positioning member corresponding portion 23 and the guide pin 2 are viewed in a plan view, the shape of an outer edge 23a of the positioning member corresponding portion 23 corresponds to the shape of an outer edge 2a of the guide pin 2. In this embodiment, these outer edges 2a and 23a include a pair of straight portions 2b extending parallel to each other and a pair of straight portions 23b extending parallel to each other, respectively as viewed in a plan view. The width between the pair of straight portions 2b and the width between the pair of straight portions 23b are set to be the same. Meanwhile, in this embodiment, the overall length (i.e. the overall length in the axial direction (longitudinal direction) of the holding portion 21) of the positioning member corresponding portion 23 is set longer than the overall length of the guide pin 2. With the above configuration, when the image guide 6 and the patient are viewed in a plan view through a radiographic device, the shape of the positioning member corresponding portion 23 matches the shape of the guide pin 2, except for the lengths.

When viewed through the radiographic device, the treatment tool corresponding portion 24 displays an image that simulates the trephine 3. The treatment tool corresponding portion 24 is formed to have an elongated rectangular flat plate shape. In this embodiment when the treatment tool corresponding portion 24 and the trephine 3 are viewed in a plan view, the shape of an outer edge 24a of the treatment tool corresponding portion 24 corresponds to the shape of an outer edge 3a of the trephine 3. In this embodiment, these outer edges 3a and 24a include a pair of straight portions 3b extending parallel to each other and a pair of straight portions 24b extending parallel to each other, respectively, as viewed in a plan view. The width between the pair of straight portions 3b and the width between the pair of straight portions 24b are set to be the same. Meanwhile, in this embodiment, the overall length (i.e. the overall length in the axial direction (longitudinal direction) of the holding portion 21) of the treatment tool corresponding portion 24 is set longer than the overall length of the trephine 3. With the above configuration, when the image guide 6 and the patient are viewed in a plan view through a radiographic device, the shape of the treatment tool corresponding portion 24 matches the shape of the trephine 3, except for the lengths.

As described above, the width of the positioning member corresponding portion 23 is set to a value that matches the width (diameter) of the guide pin 2, and the width of the treatment tool corresponding portion 24 is set to a value that matches the width (diameter) of the trephine 3. Due to this setting of the widths, the width of the positioning member corresponding portion 23 is smaller than the width of the treatment tool corresponding portion 24 as viewed in a plan view. Meanwhile, the overall length of the positioning member corresponding portion 23 and the overall length of the treatment tool corresponding portion 24 are set greater than the overall length of the guide pin 2 and the overall length of the trephine 3, respectively. With this configuration, when the image guide 6 is viewed through a radiographic device in a state where the image guide 6 is held outside the patient's body, the paths that the guide pin 2 and the trephine 3 will advance can be visually checked more clearly by the surgeon. Although, in this embodiment, the length of the positioning member corresponding portion 23 from the connecting portion 22 is set to be the same as the length of the treatment tool corresponding portion 24 from the connecting portion 22, these lengths may be different.

The configuration of the bone treatment guide set 1 is as described above. Next, a description will be given of an example of a surgical procedure of treatment of femoral head osteonecrosis using the bone treatment guide set 1.

FIG. 6 is a flowchart showing an example of a surgical procedure using the bone treatment guide set 1 (i.e. a method for using the bone treatment guide set 1). In the following description, when a description is given with reference to the flowchart, diagrams other than the flowchart will also be referenced as appropriate.

The following description will be given based on a premise that, as preparation at a stage before the bone treatment guide set 1 is used, preparation for bringing the bone treatment guide set 1 into contact the lateral part 102 of the femur 101 has been completed by for example, cutting tissues on the outer side of the patient's femur 101. Furthermore, the following description will be given based on a premise that the radiographic device, such as an X-ray device, is arranged to image the hip joint of the patient who is placed on an operating table, from the front of (i.e. from above) the patient.

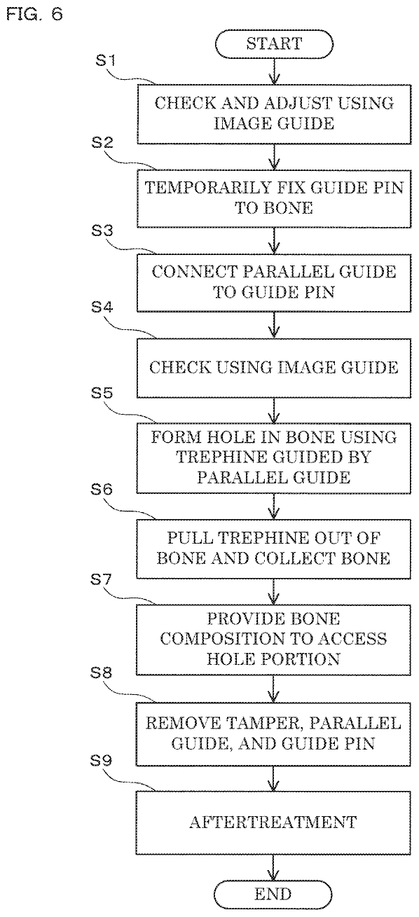

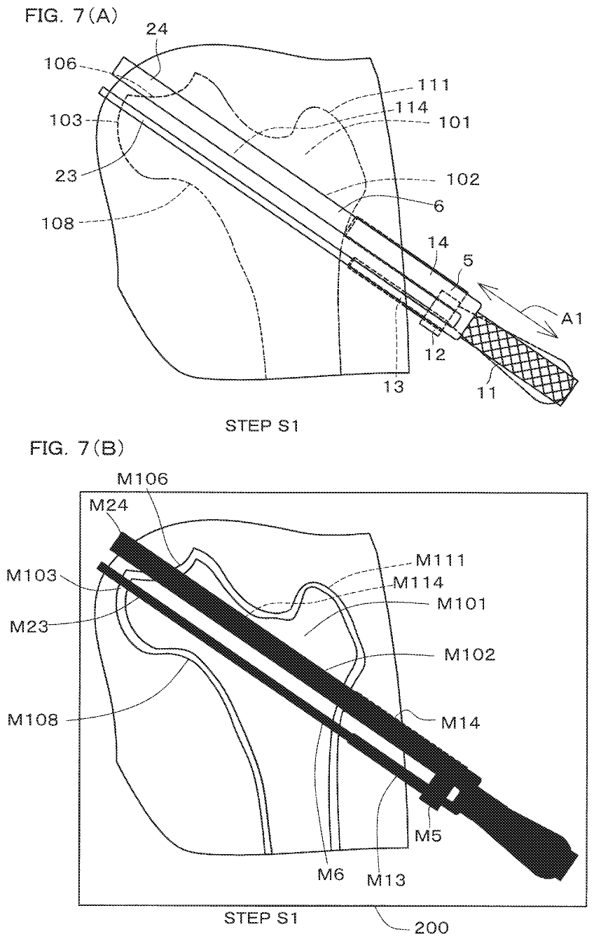

A method of using the bone treatment guide set 1 will be described with reference to FIGS. 6, 7(A), and 7(B). Initially, to determine the position at which and the direction in which the guide pin 2 is to be inserted into the femur 102, i.e. the path of the guide pin 2, the surgeon holds the image guide 6 in front of (above) the patient, checks the position of the image guide 6 relative to the femur 101 through the radiographic device, and adjusts the direction (step S1). FIG. 7(A) illustrates step S1 and is a plan view of a main part showing a state where the image guide 6 is held in front of the patient. FIG. 7(B) illustrates step S1 and shows an image appearing on a monitor 200 of the radiographic device. The result of imaging the state shown in FIG. 7(A) using the radiographic device is displayed as the image shown in FIG. 7(B). In FIGS. 7(A) and 7(B), the leading end of the positioning guide portion 13 and the leading end of the treatment tool guide portion 14 of the parallel guide 5 are arranged on the lower side of the greater trochanter 111 of the lateral part 102 of the femur 101. Step S1 is an example of a first check step according to the present invention. Note that, in the following description, images of respective members projected on the monitor 200 will be denoted by corresponding reference numerals with M at the front. In step S1, the surgeon places an image guide image M6 on the monitor 200 over a femur image M101 by changing the position and the orientation of the image guide 6. Thus, a positioning member corresponding portion image M23 in the image guide image M6 indicates the position at which the guide pin 2 will enter the inside of the femur 101, and the entering direction. Thus, the path of the guide pin 2 can be checked using the positioning member corresponding portion 23 of the image guide 6. The surgeon operates the image guide 6 such that the positioning member corresponding portion image M23 is placed at a target position and in a target entering direction of the guide pin 2.

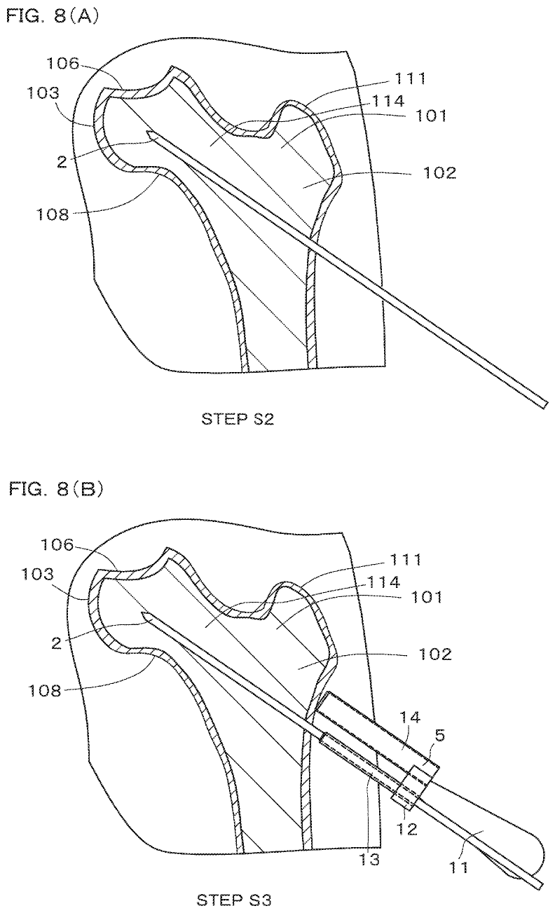

Next, a position setting step according to the present invention will be described with reference to FIGS. 6 and 8(A). The following steps S2 and S3 are examples of the position setting step according to the present invention. Initially; the surgeon arranges the guide pin 2 at the position that has been checked using the positioning member corresponding portion 23 of the image guide 6, inserts the guide pin 2 from below the greater trochanter 111 of the lateral part 102 of the femur 101 toward the medial part 108 over the checked path, and temporarily fixes the guide pin 2 (step S2). Next, the surgeon connects the parallel guide 5 to the guide pin 2 (step S3), as shown in FIG. 8(B). Through the procedure of these steps S2 and S3, the surgeon can set the position of the parallel guide 5 relative to the femur 101 while connecting the guide pin 2 inserted into the femur 101 to the positioning guide portion 13 of the parallel guide 5. Note that the positioning guide portion 13 of the parallel guide 5 may be in a state of having been put on the guide pin 2 when the guide pin 2 is being temporarily fixed to the femur 101, or may be put on the guide pin 2 after the guide pin 2 has been temporarily fixed to the femur 101. It is preferable to put the positioning guide portion 13 on the guide pin 2 after the guide pin 2 has been temporarily fixed, from the viewpoint of visibility when the guide pin 2 is temporarily fixed.

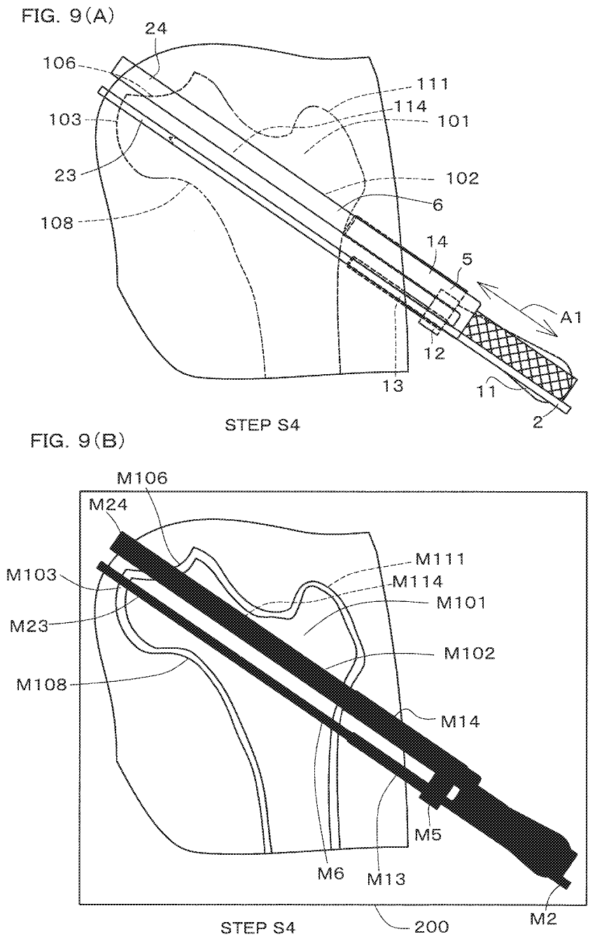

Next, a second check step according to the present invention will be described with reference to FIGS. 6, 9(A), and 9(B). The following step S4 is an example of the second check step according to the present invention. Initially, the surgeon holds the image guide 6 in front of the patient and checks the path of the trephine 3, while referencing the position and the direction of the guide pin 2 that has been temporarily fixed to the femur 101 (step S4). At this time, checking may also be performed using the radiographic device. FIG. 9(A) illustrates step S4 and is a plan view of a main part showing a state where the image guide 6 is held in front of the patient. FIG. 9(B) illustrates step S4 and shows an image appearing on a monitor 200 of the radiographic device. The result of imaging the state shown in FIG. 9(A) using the radiographic device is displayed as the image shown in FIG. 9(B). In step S4, the surgeon changes the position and the orientation of the image guide 6 such that the trephine 3 passes through the center portion of the femoral neck 114 from the lateral part 102 of the femur 101 and reaches the collapsed portion 106, and the surgeon then determines the arrangement of the trephine 3, specifically the direction and the leading end position of the treatment tool guide portion 14, while referencing the position and the orientation of the image guide 6. If necessary the surgeon references a treatment tool corresponding portion image M24 in the image guide image M6 on the monitor 200 and checks the path of the trephine 3 using the treatment tool corresponding portion 24.

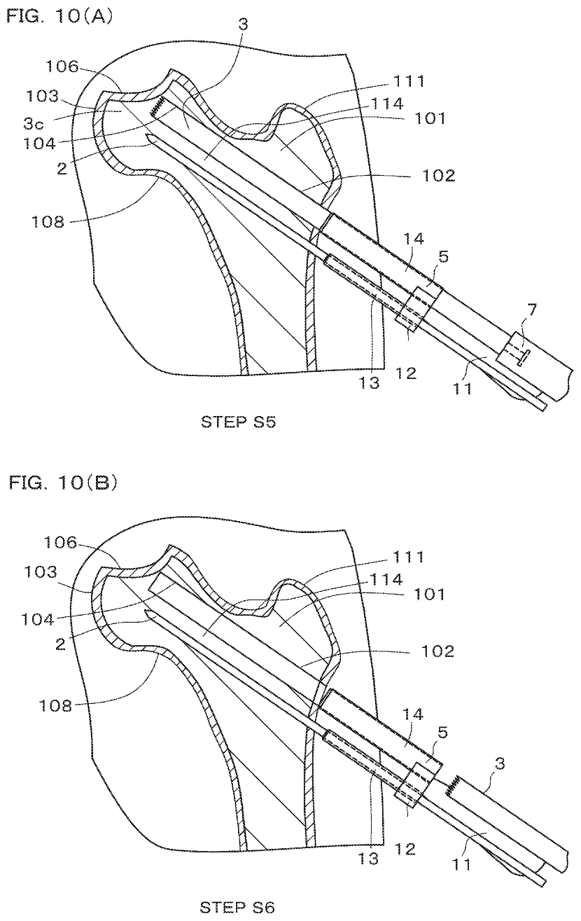

Next, a bone collection step according to the present invention will be described with reference to FIGS. 6, 10(A), and 10(B). Steps S5 and S6 are examples of the bone collection step according to the present invention, FIG. 10(A) illustrates step S5, and FIG. 10(B) illustrates step S6. Initially the surgeon fixes the trephine 3 to the drill 7, which may be an electric drill or the like. Next, the leading end of the trephine 3 is inserted into the treatment tool guide portion 14 of the parallel guide 5, whose direction and position have been determined in the aforementioned position setting step. The surgeon causes the trephine 3, which is guided by the parallel guide 5, to pass between a lower portion of the greater trochanter 111 and the femoral neck 114 in the lateral part 102 of the femur 101, while rotating the trephine 3 using the drill 7, and forms a hole extending to the collapsed portion 106 of the femoral head 103 (step S5). Through this operation, a portion of the femur 101 is taken into the trephine 3.

Next, the surgeon collects bone by pulling out the trephine 3 from the femur 101 (FIG. 10(B), step S6). Thus, in step S5, the trephine 3 is caused to enter the inside of the femur 101 to collect a portion of the femur 101 using the trephine 3, while being guided by the treatment tool guide portion 14 of the parallel guide 5.

Next, an introduction step according to the present invention will be described with reference to FIGS. 6 and 11(A). In the introduction step, the surgeon introduces the bone composition 109 into the bone through the hole portion formed in the bone in the bone collection step. Step S7 is an example of the introduction step according to the present invention, and is a step of introducing the bone composition 109 into the femur 101 through the access hole portion 104 formed in the femur 101 using the trephine 3. The bone composition 109 may be only the autologous bone 105 that has been collected using the trephine 3, or may be a mixture of the autologous bone 105 and a medical composition 8. Examples of the medical composition 8 may include a gel, a gel containing a growth factor, a medicine, an artificial bone composition, and so on. Examples of the artificial bone composition may include hydroxyapatite, and examples of the growth factor may include basic fibroblast growth factors (FGFs), bone morphogenetic proteins (BMPs), transforming growth factors (TGFs), and so on. The gel containing a growth factor may be one that gradually releases a growth factor over a long period, e.g. over about two weeks as with a bFGF slow-release gelatin gel.

Examples of the method for introducing the bone composition 109 may include a method of inserting collected bone without changing the shape thereof a method of inserting collected bone without changing the shape thereof and also inserting an artificial bone composition or the like and filling the affected area, a method of cutting a collected circular column-shaped bone into short circular column-shaped pieces and sequentially inserting these circular column-shaped bone pieces, a method of inserting a collected bone piece, then inserting a medical composition such as an artificial bone composition or a growth factor, and further inserting a bone piece, and so on.

The gel preferably is in the shape of a small piece that can be inserted into the access hole portion 104. The autologous bone 105 may be in a state where a large mass thereof has been crushed as appropriate and is in a state of not being compressed, when being inserted into the access hole portion 104. Also, the autologous bone 105 may also be crushed as appropriate, and a bone composition 109 obtained by sufficiently mixing the crushed autologous bone 105 with an artificial bone composition, a growth factor, a gel, or the like may be inserted.

The bone composition 109 is pushed to the distal side of the access hole portion 104 by the tamper 4, and is thus disposed near the collapsed portion 106. At this time, the surgeon may return the bone tissue at the collapsed portion 106 to an area in which this bone was present before collapsing, or to an area near the area in which this bone was present before collapsing, by pushing up the collapsed portion 106 using the tamper 4. It is preferable to guide the tamper 4 toward the entering position and in the entering direction through the treatment tool guide portion 14.

After the bone composition 109 has been supplied to a region near the collapsed portion 106 as shown in FIGS. 6 and 11(B), the tamper 4, the parallel guide 5, and the guide pin 2 are removed from the femur 101 (step S8). Then, aftertreatment, such as suturing an incised portion of the patient's skin, is performed, (step S9), and the surgery is completed.

As described above, according to this embodiment the parallel guide 5 has the positioning guide portion 13 and the treatment tool guide portion 14 that is arranged in an area different from the positioning guide portion 13. According to this configuration, the guide pin 2 is temporarily fixed to the bone through a manual operation or the like by the surgeon. Next, the orientation and the position of the parallel guide 5 relative to the guide pin 2 are set by connecting the positioning guide portion 13 to the guide pin 2. That is to say, the parallel guide 5 is accurately positioned relative to the femur 101. Thus, appropriate guidance regarding the direction and the leading end position of the treatment tool guide portion 14 of the parallel guide 5 can be provided relative to the femur 101. As a result, the treatment tool guide portion 14 can orient the trephine 3 toward an accurate position and in an accurate advancing direction relative to the femur 101, and a hole can be formed at a desired portion of the femur 101 with high positional accuracy using the trephine 3. Furthermore, the position of the positioning guide portion 13 differs from the position of the treatment tool guide portion 14. Thus, the guide pin 2 that is connected to the positioning guide portion 13 and the trephine 3 that is guided by the treatment tool guide portion 14 can be arranged in different areas, and accordingly the portion of the femur 101 to be collected using the trephine 3 will not have a fragile shape, such as a donut shape, due to being affected by the guide pin 2, and the amount of bone collected using the trephine 3 can also be increased.

According to this embodiment, the shape of the positioning guide portion 13 differs from the shape of the treatment tool guide portion 14. According to this configuration, the surgeon can be prevented from mistaking the positioning guide portion 13 for the treatment tool guide portion 14.

According to this embodiment, both the positioning guide portion 13 and the treatment tool guide portion 14 are formed to have a cylindrical shape, and the diameter of the positioning guide portion 13 is set smaller than the diameter of the treatment tool guide portion 14. According to this configuration, since the diameter of the positioning guide portion 13 can be made small, the diameter of the guide pin 2 can also be made small. Accordingly the diameter of the hole formed in the femur 101 by the guide pin 2 can be made smaller.

According to this embodiment, in the parallel guide 5, the positioning guide portion 13, the treatment tool guide portion 14, and the holding portion 11 are connected to each other via the connecting portion 12. Thus, as a result of the surgeon changing the position of the holding portion 11 while holding the holding portion 11, the positions of the positioning guide portion 13 and the treatment tool guide portion 14 can also be changed at the same time.

According to this embodiment, the image guide 6 has the positioning member corresponding portion 23 and the treatment tool corresponding portion 24. According to this configuration, by holding the positioning member corresponding portion 23 and the treatment tool corresponding portion 24 over the surface of the patient, for example, the surgeon can envision the arrangement of the guide pin 2 and the trephine 3 for when the guide pin 2 and the trephine 3 have entered the patient's femur 101. As a result, the surgeon can more accurately arrange the guide pin 2 and the trephine 3 relative to the patient's femur 101. Specifically, in the case where the image guide 6 is displayed on the monitor 200 of the radiographic device, a state where the patient's femur 101 and the image guide 6 overlap each other is displayed on the monitor 200, and thus, the surgeon can more accurately understand the paths of the guide pin 2 and the trephine 3.

According to this embodiment, the shape of the outer edge 23a of the positioning member corresponding portion 23 corresponds to the shape of the outer edge 2a of the guide pin 2, and the shape of the outer edge 24a of the treatment tool corresponding portion 24 corresponds to the shape of the outer edge 3a of the trephine 3. According to this configuration, by displaying the positioning member corresponding portion 23 and the treatment tool corresponding portion 24 on the monitor 200 of the radiographic device, the surgeon can more accurately envision the positions of the guide pin 2 and the trephine 3 for when the guide pin 2 and the trephine 3 have entered the patient's femur 101.

According to this embodiment, in step S1 of adjusting the position of the parallel guide 5 while referencing the image guide 6, the surgeon envisions a predicted position of the guide pin 2 relative to the femur 101 using an arrangement image of the guide pin 2 indicated by the positioning member corresponding portion 23. The surgeon then determines the arrangement of the guide pin 2 by referencing this image. Furthermore, in steps S2 and S3 of temporarily fixing the guide pin 2 to the femur 101 and connecting the parallel guide 5 to the guide pin 2, the surgeon connects the positioning guide portion 13 of the parallel guide 5 to the guide pin 2, and thus can position the parallel guide 5 at an accurate position relative to the femur with 101. Next, in step S4 of checking the path of the trephine 3, the surgeon envisions a predicted path of the trephine 3 relative to the femur 101, using an arrangement image of the trephine 3 indicated by the treatment tool corresponding portion 24. The surgeon can then cause the trephine 3 to enter the inside of the femur 101 and form a hole at a portion of the femur 101 using the trephine 3 while guiding the trephine 3 using the treatment tool guide portion 14, using this image as a reference. Thus, the surgeon can form a hole at an accurate position at a desired portion of the patient's femur 101, and keep the guide pin 2 from affecting the formation of the hole using the trephine 3, by using the parallel guide 5 and the image guide 6.

According to this embodiment, step S7 of introducing the bone composition 109 into the femur 101 through the access hole portion 104 formed in the femur 101 is provided. According to this configuration, the surgeon can introduce the bone composition 109 into an appropriate area through the access hole portion 104 formed in the femur 101. As a result, the collapsed portion 106 of the femur 101 can heal more quickly.

According to this embodiment, unlike in a configuration in which a bone different from the femur 101, such as a patient's ilium, is collected, a portion of the patient's femur 101 itself is collected as autologous bone 105, and the bone composition 109 that contains this autologous bone 105 is implanted into the collapsed portion 106. With this configuration, a bone that is present on a route (access hole portion 104) along which the bone is to be implanted can be used, as-is, as the autologous bone 105. Accordingly collection and implantation of the bone can be performed simultaneously.

This embodiment employs a configuration in which the collapsed portion 106 of the femoral head 103 is raised using the tamper 4. With this configuration, the collapsed femoral head 103 can be restored to its original shape by physically applying pressure to the collapsed portion 106 using the tamper 4.

As described above, according to this embodiment, bone grafting can be performed more securely more accurately, and more simply.

Although an embodiment of the present invention has been described thus far, the present invention is not limited to the above-described embodiment, and various modifications can be made within the scope stated in the claims. For example, the following modifications are possible.

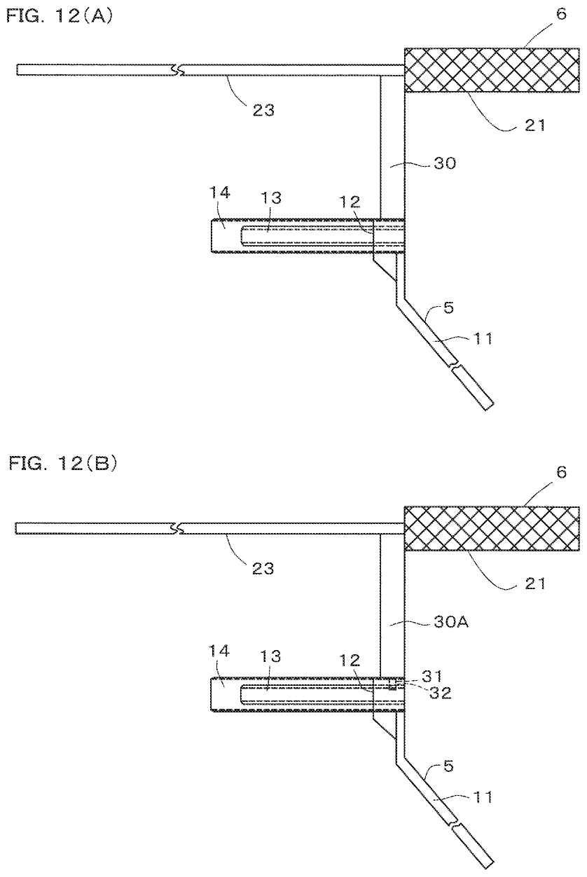

(1) The above embodiment has described, as an example, a mode in which the parallel guide 5 and the image guide 6 are separate members. However, this need not be the case. For example, the parallel guide 5 and the image guide 6 may be formed as an integrated member, as shown in FIG. 12(A). In a modification shown in FIG. 12(A), the parallel guide 5 and the image guide 6 are fixed to each other via a connecting bar 30. In this case, an end of the connecting bar 30 is fixed to the connecting portion 12 of the parallel guide 5 by means of welding or the like, and the other end of the connecting bar 30 is fixed to the holding portion 21 of the image guide 6 by means of welding or the like. Also, the positioning member corresponding portion 23 and the treatment tool corresponding portion 24 of the image guide 6 are arranged so as to overlap the positioning guide portion 13 and the treatment tool guide portion 14, respectively, as viewed in a plan view. The leading end position of the positioning member corresponding portion 23 and the leading end position of the treatment tool corresponding portion 24 of the image guide 6 are set in areas farther from the connecting bar 30 than the leading end position of the positioning guide portion 13 and the leading end position of the treatment tool guide portion 14, respectively. With this configuration, the surgeon can simultaneously hold the parallel guide 5 and the image guide 6, and accordingly can handle the parallel guide 5 more readily. Furthermore, since the relative positions of the parallel guide 5 and the image guide 6 are fixed, time and effort involved in adjusting the relative positions of the parallel guide 5 and the image guide 6 can be saved.

(2) Note that the parallel guide 5 and the image guide 6 may be connected to each other using a connecting bar 30A, as shown in FIG. 12(B). That is to say the bone treatment guide set 1 may further include the connecting bar 30A for connecting the parallel guide 5 to the image guide 6, and the parallel guide 5 and the image guide 6 may be detachably connected to each other via the connecting bar 30A Note that, in the example shown in FIG. 12(B), the connecting bar 30A is fixed to the holding portion 21 of the image guide 6, and a fitting protruding portion 31, whose cross-sectional area is smaller than that of the connecting bar 30A is provided at a leading end thereof. The fitting protruding portion 31 can be fitted to a recessed portion 32, which is formed in a top surface of the connecting portion 12 of the parallel guide 5. With this configuration, when the image guide 6 is needed, the image guide 6 can be connected to the parallel guide 5 using the function of the connecting bar 30A through the connection between the fitting protruding portion 31 and the recessed portion 32. Meanwhile, if the image guide 6 is not needed, the parallel guide 5 can be used alone by removing the fitting protruding portion 31 from the recessed portion 32. Note that FIG. 12(B) shows an example mode in which the connecting bar 30A is fixed to the image guide 6 and is fitted to the parallel guide 5, and thus the parallel guide 5 and the image guide 6 are connected to each other via the connecting bar 30A, but this need not be the case. A mode may alternatively be implemented in which the parallel guide 5 and the image guide 6 are connected to each other via the connecting bar 30A due to the connecting bar 30A being fixed to the parallel guide 5 and fitted to the image guide 6. Alternatively a mode may be implemented in which the parallel guide 5 and the image guide 6 are connected to each other via the connecting bar 30A due to the connecting bar 30A being fitted to both the parallel guide 5 and the image guide 6. In any of the above modes, when the image guide 6 is needed, the parallel guide 5 and the image guide 6 can be connected by the connecting bar 30A. Meanwhile, if the image guide 6 is not needed, the parallel guide 5 can be used alone by canceling the connection between the parallel guide 5 and the image guide 6 via the connecting bar 30A and removing the image guide 6 solely or together with the connecting bar 30A.

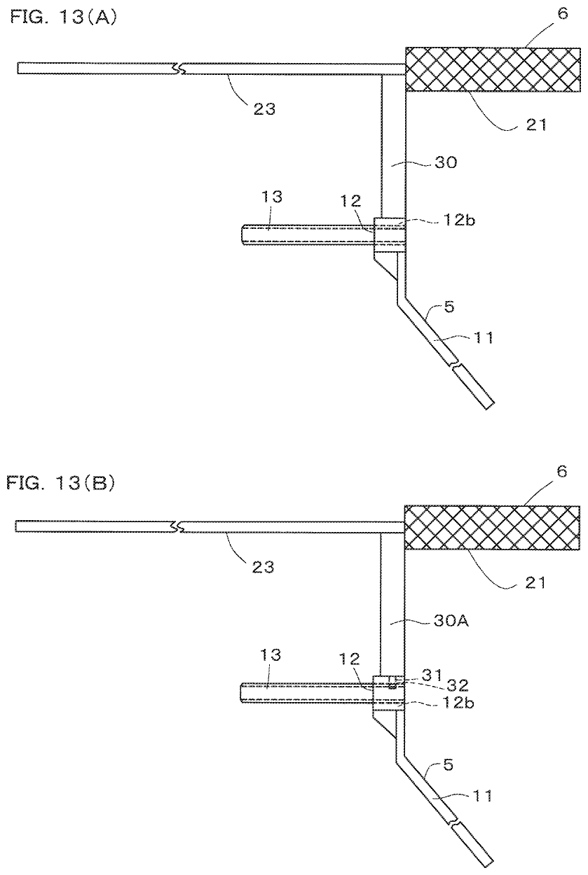

(3) In the configuration shown in FIG. 12(A) in which the parallel guide 5 and the image guide 6 are formed as an integrated member, the treatment tool guide portion 14 may be omitted as shown in FIG. 13(A). In this case, for example, the joint face 12b itself of the connecting portion 12 functions as the treatment tool guide portion. Also, in the configuration shown in FIG. 12(B) in which the parallel guide 5 and the image guide 6 are connected to each other using the connecting bar 30A, the treatment tool guide portion 14 may be omitted as shown in FIG. 13(B). In this case as well, for example, the joint face 12b itself of the connecting portion 12 functions as the treatment tool guide portion.

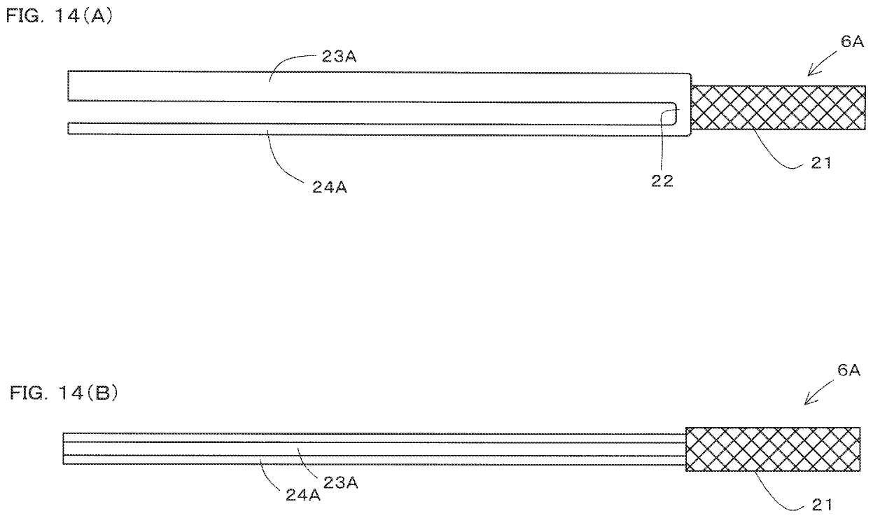

(4) The above embodiment has described, as an example, a mode in which the image guide 6 has a flat plate shape. However, this need not be the case. For example, a configuration may be employed in which the shape of a positioning member corresponding portion 23A of an image guide 6A is formed to have a circular column shape or a cylindrical shape similar to the shape of the guide pin 2, and the shape of a treatment tool corresponding portion 24A is formed to have a cylindrical shape or a circular column shape similar to the shape of the trephine 3, as shown in FIGS. 14(A) and 14(B). In the case where the image guide 6A is used, an image guide image and a bone treatment guide image projected on the monitor 200 of the radiographic device can be made more similar to each other when, for example, the patient is viewed from a side, in addition to when the patient is viewed in a plan view.

(5) The above embodiment has described, as an example, a mode in which the parallel guide 5 and the guide pin 2 are separate members. However, this need not be the case. For example, the guide pin 2 inserted into the positioning guide portion 13 of the parallel guide 5 may be fixed to the positioning guide portion 13 by means of welding or the like. Also, the positioning guide portion 13 and the guide pin 2 may be molded integrally. In this case, the parallel guide 5 and the guide pin 2 need not be operated separately and the operation to handle the bone treatment guide set 1 performed by the surgeon can be made simpler.

(6) The above embodiment has described, as an example, a mode in which one type of parallel guide 5 is used. However, this need not be the case. For example, multiple types of treatment tool guide sets that have guide pins with different outside diameters, trephines with different outside diameters, positioning guide portions with different inside diameters, and treatment tool guide portions with different inside diameters may be provided in accordance with the physique of patients. In this case, the treatment tool guide sets have multiple types of guide pins with different outside diameters, multiple types of trephines with different outside diameters, and multiple types of treatment tool guides with different inside diameters of the positioning guide portions and different inside diameters of the treatment tool guide portions.

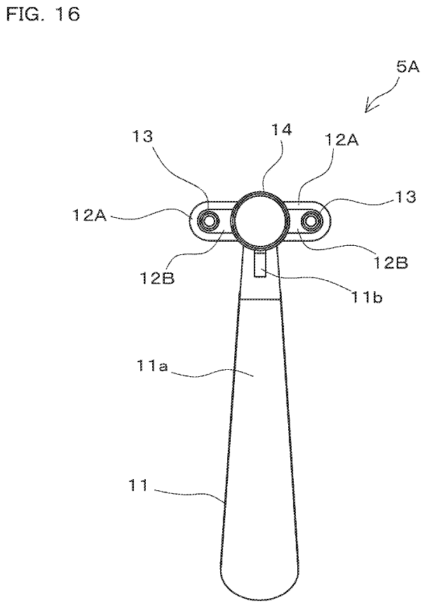

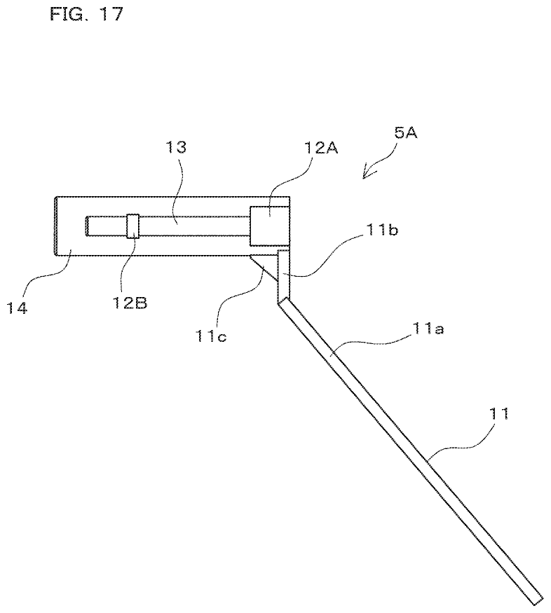

(7) The above embodiment has described, as an example, a mode of the parallel guide (bone treatment guide) 5 that includes one positioning guide portion 13 and one treatment tool guide portion 14. However, this need not be the case. For example, a mode of a parallel guide (bone treatment guide) 5A, which includes a pair of positioning guide portions 13, may alternatively be implemented, as shown in FIGS. 15 to 17. Note that FIG. 15 is a plan view of the parallel guide 5A, FIG. 16 is a front elevational view of the parallel guide 5A, and FIG. 17 is a side view of the parallel guide portion 5A.

In the modification shown in FIGS. 15 to 17, the parallel guide 5A is provided with only one treatment tool guide portion 14, and is provided with a pair of positioning guide portions 13. The pair of positioning guide portions 13 are integrally connected to the treatment tool guide portion 14 on the respective sides thereof in the width direction W1 perpendicular to the longitudinal direction (axial direction) of the treatment tool guide portion 14. The pair of positioning guide portions 13 are integrally connected to the treatment tool guide portion 14 via first connecting portions 12A and second connecting portions 12B on the respective sides of the treatment tool guide portion 14 in the width direction W1. Note that the first connecting portions 12A integrally connect base end portions of the positioning guide portions 13 to the base end portion of the treatment tool guide portion 14. The second connecting portions 12B integrally connect portions on the leading end side of the positioning guide portions 13 to a portion on the leading end side of the treatment tool guide portion 14. The positioning guide portions 13 are arranged next to the treatment tool guide portion 14 in the width direction W1, and are connected to the treatment tool guide portion 14 via the first connecting portions 12A and the second connecting portions 12B, in a state of extending parallel to the treatment tool guide portion 14.

In the configuration in the modification shown in FIGS. 15 to 17, the positioning guide portions 13 are connected on both sides of the treatment tool guide portion 14 in the width direction W1. Accordingly, the surgeon can select and use one of the pair of positioning guide portions 13 provided on the respective sides of the treatment tool guide portion 14 in the width direction W1 when connecting a positioning guide portion 13 to the guide pin 2, which is the positioning member. For this reason, the same parallel guide 5A can also be used in the case where the guide pin 2 is temporarily fixed to the femur 101 at a position on either one of two sides, in the width direction W1, of the trephine 3 that is to enter the inside of the femur 101, relative to the position at which the trephine 3, which is a treatment tool, is to enter the inside of the femur 101. Accordingly the same parallel guide 5A can also be used in the case where the parallel guide 5A is used in surgery on either a left leg or a right leg.

Note that the modification shown in FIGS. 15 to 17 has described an example mode in which a pair of positioning guide portions 13 are provided, and the pair of positioning guide portions 13 are connected to the treatment tool guide portion 14 on the respective sides of the treatment tool guide portion 14 in the width direction W1. However, this need not be the case. For example, a mode of a parallel guide (bone treatment guide) may alternatively be employed in which a pair of treatment tool guide portions 14 are provided, and the pair of treatment tool guide portions 14 are connected to a positioning guide portion 13 on the respective sides of the positioning guide portion 13 in the width direction W1.

(8) A mode may also be employed in which a parallel guide (bone treatment guide) 5A further includes a tubular replaceable member 40, which is replaceably attached to the treatment tool guide portion 14 by being detachably inserted into the treatment tool guide portion 14, as shown in FIG. 15. The replaceable member 40 is made of a resin material, such as polyethylene, and is formed to have a cylindrical shape, for example. The replaceable member 40 is inserted into the treatment tool guide portion 14, fitted into an inner-circumferential portion 14a of the treatment tool guide portion 14, and is attached to the treatment tool guide portion 14. The trephine 3, which is a treatment tool, is inserted into the replaceable member 40 attached to the treatment tool guide portion 14, and is thus guided toward the position at which the trephine 3 is to enter the inside of the femur 101, and in the entering direction. That is to say the trephine 3 is guided to enter the inside of the femur 101 by being inserted into an inner-circumferential portion 40a of the replaceable member 40 fitted to the inner-circumferential portion of the treatment tool guide portion 14. Note that the replaceable member 40 may be provided as a disposable member, for example, and may be replaced every time the parallel guide (bone treatment guide) 5A is used.

In the configuration in the modification shown in FIG. 15, when the trephine 3 is guided to enter the inside of the femur 101 by the treatment tool guide portion 14, the trephine 3 is inserted into the replaceable member 40 attached to the treatment tool guide portion 14. Thus, the trephine 3 can be prevented from coming into direct contact with the treatment tool guide portion 14 and causing damage, such as wear. Also, even if the replaceable member 40 is damaged, e.g. is worn when the parallel guide 5A is used, only the replaceable member 40 can be readily replaced. Accordingly the treatment tool guide portion 14 can be repeatedly used simply by replacing the replaceable member 40, without damaging the treatment tool guide portion 14.

(9) The above embodiment has described, as an example, a mode of treating femoral head osteonecrosis after a portion of the femoral head 103 has collapsed, but this need not be the case. For example, the bone treatment guide set 1 may also be used when osteonecrosis of the femur is treated in a state before the femoral head 103 has collapses.

(10) The above embodiment has described, as an example, a mode of treating a disease of the hip joint (femoral head 103). However, this need not be the case. The bone treatment guide set according to the present invention may also be used in bone treatment in other parts, including treatment of a humerus (arm joint), such as the head of a humerus, a foot (foot joint), a knee joint including the distal part of a femur and the proximal part of a tibia, and so on.

(11) Although an embodiment of the present invention has been described thus far, the present invention need only have at least a parallel guide, and at least some of the other members need not be included in the bone treatment guide set.

### INDUSTRIAL APPLICABILITY

The present invention can be widely applied as a bone treatment guide, a bone treatment guide set, and a method for using a bone treatment guide.

### DESCRIPTIONS OF REFERENCE NUMERALS

1 Bone treatment guide set
2 Guide pin (positioning member)
2a Outer edge of guide pin (outer edge of positioning member)
3 Trephine (treatment tool)
3a Outer edge of trephine (outer edge of treatment tool)
5 Parallel guide (bone treatment guide)
6,6A Image guide
11 Holding portion
12 Connecting portion
13 Positioning guide portion
14 Treatment tool guide portion
23,23A Positioning member corresponding portion
23a Outer edge of positioning member corresponding portion
24,24A Treatment tool corresponding portion
24a Outer edge of treatment tool corresponding portion
101 Femur (bone of patient)
104 Access hole portion (hole portion formed in bone)
109 Bone composition
D13 Inside diameter (diameter) of positioning guide portion
D14 Inside diameter (diameter) of treatment tool guide portion

1: A bone treatment guide comprising:
a positioning guide portion that is to be connected to a positioning member, which is to be temporarily fixed to a bone of a patient; and
a treatment tool guide portion configured to be able to guide a treatment tool for treating the bone toward a position at which the treatment tool is to enter inside of the bone and in an entering direction, the treatment tool guide portion being arranged in an area different from the positioning guide portion.

2: The bone treatment guide according to claim 1,
wherein a shape of the positioning guide portion differs from a shape of the treatment tool guide portion.

3: The bone treatment guide according to claim 2,
wherein both the positioning guide portion and the treatment tool guide portion are formed to have a cylindrical shape, and
a diameter of the positioning guide portion is set smaller than a diameter of the treatment tool guide portion.

4: The bone treatment guide according to claim 1, further comprising:
a holding portion to be held by a surgeon; and
a connecting portion that is connected to the positioning guide portion, the treatment tool guide portion, and the holding portion.

5: The bone treatment guide according to claim 1,
wherein a pair of the positioning guide portions are provided, and
the pair of positioning guide portions are connected to the treatment tool guide portion on respective sides of the treatment tool guide portion in a width direction perpendicular to a longitudinal direction of the treatment tool guide portion.

6: The bone treatment guide according to claim 1, further comprising
a replaceable member having a tubular shape that is replaceably attached to the treatment tool guide portion by being detachably inserted into the treatment tool guide portion that is formed to have a tubular shape,
wherein the treatment tool is guided toward the position at which the treatment tool is to enter the inside of the bone and in the entering direction, by being inserted into the replaceable member attached to the treatment tool guide portion.

7: A bone treatment guide set comprising:
the bone treatment guide according to claim 1; and
an image guide,
wherein the image guide includes:
a positioning member corresponding portion for indicating an arrangement of the positioning member; and
a treatment tool corresponding portion for indicating an arrangement of the treatment tool.

8: The bone treatment guide set according to claim 7,
wherein a shape of an outer edge of the positioning member corresponding portion corresponds to a shape of an outer edge of the positioning member, and
a shape of an outer edge of the treatment tool corresponding portion corresponds to a shape of an outer edge of the treatment tool.

9: The bone treatment guide set according to claim 7,
wherein the bone treatment guide and the image guide are fixed to each other and are formed into an integrated member.

10: The bone treatment guide set according to claim 7, further comprising
a connecting bar for connecting the bone treatment guide and the image guide to each other,
wherein the bone treatment guide and the image guide are detachably connected to each other via the connecting bar.

11: A method for using a bone treatment guide comprising:
a first check step of checking a path of a positioning member using a positioning member corresponding portion of an image guide that includes: the positioning member corresponding portion for indicating an arrangement of the positioning member, which is to be temporarily fixed to a bone of a patient; and a treatment tool corresponding portion for indicating an arrangement of a treatment tool for treating the bone;
a position setting step of setting a position of the bone treatment guide according to claim 1 relative to the bone while connecting the positioning member that has been temporarily fixed to the bone to the positioning guide portion of the bone treatment guide;
a second check step of checking a path of the treatment tool using the treatment tool corresponding portion of the image guide; and
a bone collection step of causing the treatment tool to enter inside of the bone while guiding the treatment tool using the treatment tool guide portion, and collecting a portion of the bone using the treatment tool.

12: The method for using a bone treatment guide according to claim 11, further comprising
an introduction step of introducing a bone composition into the bone through a hole portion formed in the bone during the bone collection step.
